(19) United States
(12) Patent Application Publication
(10) Pub. No.: US 2013/0004556 A1
(43) Pub. Date: Jan. 3, 2013

(54) CHEWABLE FOAMS FOR COSMETIC PRODUCTS

(75) Inventors: Steffen HOFACKER, Odenthal (DE); Thorsten RISCHE, Unna (DE); Meike NIESTEN, Koeln (DE); Thomas FELLER, Solingen (DE); Thomas MICHAELIS, Leverkusen (DE)

(73) Assignee: BAYER MATERIALSCIENCE AG, Leverkusen (DE)

(21) Appl. No.: 13/612,939

(22) Filed: Sep. 13, 2012

Related U.S. Application Data
(62) Division of application No. 11/787,455, filed on Apr. 17, 2007.

(30) Foreign Application Priority Data
Apr. 22, 2006 (DE) 102006018826.8
Apr. 26, 2006 (DE) 102006019742.9

(51) Int. Cl. A61K 8/87; A61K 8/02; A61Q 11/00; A61K 8/21

(52) U.S. Cl. 424/401; 424/49; 424/52

(57) ABSTRACT

The invention relates to novel chewable foams which are useful in the oral care sector and which are based on polyurethane-polyureas, to a method for the production of these novel chewable foams, and also to use thereof.

### CROSS-REFERENCE TO RELATED PATENT APPLICATIONS

This application is a Divisional Application of U.S. application Ser. No. 11/787,455 filed Apr. 17, 2007, which claims priority to German Application No. 102006019742.9 filed Apr. 26, 2006 and German Application No. 102006018826.8 filed Apr. 22, 2006, each hereby incorporated by reference in their entireties.

### BACKGROUND OF THE INVENTION

The present invention relates to novel chewable foams for the oral care sector which are based on polyurethane-polyureas, to a method for their production and also to the use of these chewable foams.

Organic polymers are widely used as raw materials in cosmetic products. They may be found in many cosmetic products such as, for example, hair sprays, hair gels, mascara, lipsticks, creams, etc. In the oral care sector, polymers may be found, for example, in the form of toothbrushes, dental flosses, etc.

Due to the developing requirement of society for oral care during the period between meals or after consumption, for example, of a between-meal snack (such as for example, sweets, nicotine, alcohol, etc.) or on account of increased mobility (for example during air or train travel) in which conventional teeth cleaning with water, toothpaste and toothbrush is not possible, various dental care products have been developed. Such previously developed products include, for example, dental care chewing gums and/or dental care wipes.

Dental care chewing gums essentially consist of gum base. The gum base in turn consists of natural or synthetic polymers such as, for example, latex, polyvinyl ethers, polyisobutylene vinyl ethers, polyisobutene, etc. Such dental care chewing gums, as dental care compositions, generally contain pH-controlling substances which thus counteract the development of tooth decay (cavities). Owing to their plastic behavior, such dental care chewing gums, however, scarcely contribute to cleaning the chewing surfaces or tooth sides. In addition, chewing gums generally have the disadvantage that they must frequently be mechanically removed from public streets and spaces due to their adhesive properties, and disposed of. This mechanical removal and disposal leads to considerable cleaning expenditure of floor and road surfaces.

Teeth wipes (such as, for example, Oral-B Brush Aways™, commercially available from Gillette GmbH & Co. OHG, Germany) are distinguished in that they achieve good cleaning action of the tooth sides by applying the teeth wipe onto a finger and by rubbing the teeth. However, the mode of employing such teeth cleaning wipes in public has gained little acceptance for aesthetic reasons, and is thus not an alternative to using a conventional toothbrush.

It has now been found that polymeric materials may be produced from special polyurethane-polyurea dispersions, in which the polymeric materials are suitable as chewable foams for the oral care sector. This is due to, among other things, the particularly advantageous mechanical properties of these polymeric materials.

### SUMMARY OF THE INVENTION

The present invention relates to chewable foams made from polyurethane-polyureas.

It is advantageous when the chewable foams have a 100% modulus of 0.1 to 8.0 MPa, at a tensile strength of 0.5 to 80 MPa and an extensibility of 100 to 3000% (determined as specified in DIN 53504 on a free film of the gum base having a sheet thickness >100 μm). It is preferred for the chewable foams to have a 100% modulus of 0.3 to 8.0 MPa, at a tensile strength of 1 to 80 MPa and an extensibility of 100 to 2500%. More preferred are those chewable foams which have a 100% modulus of 0.3 to 6.0 MPa at a tensile strength of 1 to 60 MPa and an extensibility of 200 to 2000%. Most preferred are those chewable foams which have a 100% modulus of 0.3 to 5.0 MPa at a tensile strength of 1 to 55 MPa and an extensibility of 300 to 1800%.

The tensile tests were carried out according to DIN 53504 using a dumbbell-shaped rod specimen S2 according to DIN 53504. The tensile moduli were determined according to DIN EN ISO 527. The layer thickness of the specimens was 2.5 mm +/−1 mm).

It is also advantageous if the ratio between the tensile strength and the modulus of elasticity of the polymeric chewable foam according to the invention is greater than or equal to 1, preferably greater than 1.5, and particularly preferably greater than 2 and the ratio of the product of the tear propagation resistance (according to DIN ISO 34-1 (2004)) and the modulus of elasticity to the square of the tensile strength is lower than 4 mm and preferably lower than 1.5 mm.

In addition, the stability of the polymeric chewable foam under compression should be greater than 50 MPa and preferably greater than 75 MPa.

The present invention also relates to a method for the production of the chewable foams according to the invention. This method comprises foaming of one or more polyurethane-polyurea dispersions (I) as described herein, and optionally, with additional components of the chewable foams, and subsequently drying the foamed material.

Suitable polyurethane-polyurea dispersions (I) for the present invention include those which are obtainable from
A) one or more isocyanate-functional prepolymers which comprise the reaction product of:
   a1) one or more organic polyisocyanates, with
   a2) one or more polymeric polyols which have number-average molecular weights of 400 to 8000 g/mol and OH functionalities of 1.5 to 6,
   a3) optionally, one or more hydroxyfunctional compounds having molecular weights of 62 to 399 g/mol, and
   a4) optionally, one or more hydroxyfunctional, ionic or potentially ionic and/or nonionic hydrophilizing agents;
      in which the free NCO groups are then in whole or in part reacted with
B) at least one compound selected from the group consisting of:
   b1) one or more aminofunctional compounds having molecular weights of 32 to 400 g/mol, and
   b2) one or more aminofunctional, ionic or potentially ionic hydrophilizing agents;
      with chain extension, and in which the prepolymers are dispersed before, during, or after the reaction with said compound B) in water, and optionally, with potentially ionic groups present which are able to be converted into the ionic form by partial or complete reaction.

### DETAILED DESCRIPTION OF THE INVENTION

As used herein, isocyanate-reactive groups include, for example, amino, hydroxyl and/or thiol groups unless otherwise stated.

Suitable organic polyisocyanates to be used as component al) include, for example, 1,4-butylene diisocyanate, 1,6-hexamethylene diisocyanate (HDI), isophorone diisocyanate (IPDI), 2,2,4- and/or 2,4,4-trimethylhexamethylene diisocyanate, the isomeric bis-(4,4′-isocyanatocyclohexyl)methanes or mixtures thereof of any desired isomer content, 1,4-cyclohexylene diisocyanate, 1,4-phenylene diisocyanate, 2,4- and/or 2,6-toluene diisocyanate, 1,5-naphthylene diisocyanate, 2,2′- and/or 2,4′- and/or 4,4′-diphenylmethane diisocyanate, 1,3- and/or 1,4-bis-(2-isocyanatoprop-2-yl)benzene (TMXDI), 1,3-bis(isocyanatomethyl)benzene (XDI), (S)-alkyl 2,6-diisocyanatohexanoates, (L)-alkyl 2,6-diisocyanatohexanoates, having branched, cyclic or acyclic alkyl groups having up to 8 carbon atoms, etc.

In addition to the abovementioned polyisocyanates, component al) can also be comprised of, some proportion of modified diisocyanates which have uretione, isocyanurate, urethane, allophanate, biuret, iminooxadiazinedione and/or oxadiazinetrione structures, and also, of unmodified polyisocyanate having more than 2 NCO groups per molecule such as, for example, 4-isocyanatomethyl-1,8-octane diisocyanate (nonane triisocyanate) or triphenylmethane-4,4′,4″-triisocyanate.

Preferably, component al) comprises polyisocyanates or polyisocyanate mixtures of the abovementioned type which contain solely aliphatically and/or cycloaliphatically bound isocyanate groups and an average NCO functionality of the mixture of 2 to 4, preferably 2 to 2.6, and more preferably 2 to 2.4.

It is most preferred that component al) comprises 1,6-hexamethylene diisocyanate, isophorone diisocyanate, the isomeric bis-(4,4′-isocyanatocyclohexyl)methanes, and mixtures thereof.

Component a2) comprises one or more polymeric polyols having number-average molecular weights of 400 to 6000 g/mol, and more preferably of from 600 to 3000 g/mol.

These polymeric polyols also typically have OH functionalities of 1.8 to 3, and more preferably from 1.9 to 2.1.

Suitable compounds to be used as the polymeric polyols a2) in accordance with the present invention include, for example, polyester polyols, polyacrylic polyols, polyurethane polyols, polycarbonate polyols, polyether polyols, polyester polyacrylic polyols, polyurethane polyacrylic polyols, polyurethane polyester polyols, polyurethane polyether polyols, polyurethane polycarbonate polyols and polyesterpolycarbonate polyols, which are known per se in polyurethane coating technology. These compounds can be used individually or in any desired mixtures with one another.

Suitable polyester polyols are the polycondensates known per se of di-, and also if appropriate, tri- and tetraols, and di-, and also if appropriate, tri- and tetracarboxylic acids or hydroxycarboxylic acids or lactones. Instead of the free polycarboxylic acids, use can also be made of the corresponding polycarboxylic anhydrides or corresponding polycarboxylic esters of lower alcohols for producing the polyester polyols.

Examples of suitable diols for preparing the polyester polyols are ethylene glycol, butylene glycol, diethylene glycol, triethylene glycol, polyalkylene glycols such as polyethylene glycol, and in addition, 1,2-propanedia, 1,3-propanediol, butane-1,3-diol, butane-1,4-diol, and also hexane-1,6-diol and various isomers of hexanediol, neopentyl glycol and hydroxypivalic neopentyl glycol ester, whereas hexane-1,6-diol and isomers of neopentyl glycol and hydroxypivalic neopentyl glycol esters are preferred. In addition, it is also possible to use polyols such as trimethylol propane, glycerol, erythritol, pentaerythritol, trimethylol benzene and/or trishydroxyethyl isocyanurate.

Some examples of suitable dicarboxylic acids includes compounds such as phthalic acid, isophthalic acid, terephthalic acid, tetrahydrophthalic acid, hexahydrophthalic acid, cyclohexanedicarboxylic acid, adipic acid, azelaic acid, sebacic acid, glutaric acid, tetrachlorophthalic acid, maleic acid, fumaric acid, itaconic acid, malonic acid, suberic acid, 2-methylsuccinic acid, 3,3-diethyl-glutaric acid and/or 2,2-dimethylsuccinic acid. Also, as an acid source, it is possible to use any of the corresponding anhydrides.

If the average functionality of the polyol to be esterified is >2, in addition, use can also be made, in conjunction with the above, of monocarboxylic acids, such as benzoic acid and hexanecarboxylic acid.

Preferred acids are aliphatic and/or aromatic acids of the abovementioned type. In particular, preference is given to adipic acid, isophthalic acid and phthalic acid.

Hydroxycarboxylic acids which can be used as reactants in the production of a suitable polyester polyol having terminal hydroxyl groups are, for example, hydroxycaproic acid, hydroxybutyric acid, hydroxydecanoic acid, hydroxystearic acid and the like. Suitable lactones are caprolactone, butyrolactone and homologues. Preference is given to caprolactone.

Other suitable polymeric polyols to be used as component a2) in accordance with the present invention include hydroxyl-containing polycarbonates, and preferably polycarbonatediols, having number-average molecular weights (Mₙ) of 400 to 8000 g/mol, and preferably 600 to 3000 g/mol. These hydroxyl-containing polycarbonates are obtainable by reaction of carbonic acid derivatives such as diphenyl carbonate, dimethyl carbonate or phosgene with polyols, and preferably with diols.

Examples of suitable diols are ethylene glycol, 1,2- and 1,3-propanediol, 1,3- and 1,4-butanediol, 1,6-hexanediol, 1,8-octanediol, neopentyl glycol, 1,4-bishydroxy- methylcyclohexane, 2-methyl-1,3-propanediol, 3-methyl-1,5-pentanediol, 2,2,4-trimethylpentane-1,3-diol, dipropylene glycol, polypropylene glycols, dibutylene glycol, polybutylene glycols, bisphenol A, tetrabromobisphenol A and lactone-modified diols of the abovementioned type may also be considered as suitable. Mixtures of different diols can also be used.

Preferably, the diol component which is reacted with a carbonic acid derivative to form the hydroxyl-containing polycarbonates contains from 40 to 100% by weight of hexanediol, with preference being given to 1,6-hexanediol and/or hexanediol derivatives. Such hexanediol derivatives are based on hexanediol and have, in addition to terminal OH groups, ester or ether groups. Such derivatives are obtainable by reaction of hexanediol with excess caprolactone, or by etherification of hexanediol with itself to give di- or trihexylene glycol.

Instead of, or in addition to, pure polycarbonatediols, polyether-polycarbonatediols can also be used as suitable polymeric polyols for component a2) in the present invention. Such polyether-polycarbonatediols contain, as diol component, in addition to the dials described, also polyetherdiols.

Hydroxyl-containing polycarbonates used herein are preferably of linear structure, but they can also contain branched points owing to the incorporation of the polyfunctional components, and in particular, low-molecular-weight polyols. Suitable substances for this include compounds such as, for example, glycerol, trimethylol propane, hexane-1,2,6-triol, butane-1,2,4-triol, trimethylol propane, trimethylolethane, pentaerythritol, quinite, mannitol, sorbitol, methyl glycoside or 1,3,4,6-dianhydrohexite.

Suitable polyether polyols for component a2) include, for example, the polytetramethylene glycol polyethers known per se in polyurethane chemistry, such as those which are obtainable by polymerization of tetrahydrofuran by means of cationic ring opening.

Likewise, other suitable polyether polyols are the addition products known per se of styrene oxide, ethylene oxide, propylene oxide, butylene oxides and/or epichlorohydrin to di- or polyfunctional starter molecules.

Suitable starter molecules for the preparation of polyether polyols include all compounds known from the prior art such as, for example, water, butyl diglycol, glycerol, diethylene glycol, trimethylol propane, propylene glycol, sorbitol, ethylenediamine, triethanolamine, 1,4-butanediol, etc.

A preferred embodiment of the invention is polyurethane dispersions (I) which contain, as part or all of component a2), a mixture of polycarbonate polyols and polytetramethylene glycol polyols. The fraction of polycarbonate polyols in the mixture is 20 to 80% by weight, and the fraction of polytetramethylene glycol polyols is 80 to 20% by weight, with the sum of these %'s by weight totalling 100% by weight of the mixture. It is more preferred to use a mixture comprising a fraction of from 30 to 75% by weight of polytetramethylene glycol polyols and from 25 to 70% by weight of polycarbonate polyols. It is most preferred to use a mixture comprising a fraction of from 35 to 70% by weight of polytetramethylene glycol polyols and 30 to 65% by weight of polycarbonate polyols, with the proviso that, in each of these mixtures, the sum of the percentages by weight of polycarbonate and polytetramethylene glycol polyols totals 100% by weight. In addition, the fraction of the sum of polycarbonate polyols and polytetramethylene glycol polyether polyols in component a2) is preferably at least 50% by weight, more preferably 60% by weight, and most preferably at least 70% by weight (based on 100% by weight of component a2)).

Suitable compounds to be used as component a3) include polyols of the above described molecular weight range (i.e. 62 to 399) which have up to 20 carbon atoms. Examples of such compounds include ethylene glycol, diethylene glycol, triethylene glycol, 1,2-propanediol, 1,3-propanediol, 1,4-butanediol, 1,3-butylene glycol, cyclohexanediol, 1,4-cyclohexanedimethanol, 1,6-hexanediol, neopentyl glycol, hydroquinone dihydroxy ethyl ether, bisphenol A (2,2-bis(4-hydroxyphenyl)propane, hydrogenated bisphenol A, (2,2-bis(4-hydroxycyclohexyl)-propane), trimethylol propane, glycerol, pentaerythritol, and also any desired mixtures thereof among one another.

Suitable compounds for a3) also include ester diols of the above described molecular weight range (i.e. 62 to 399) such as, for example, α-hydroxybutyl ε-hydroxycaproate, ω-hydroxyhexyl γ-hydroxybutyrate, β-hydroxyethyl adipate and/or bis((3-hydroxyethyl) terephthalate.

In addition, component a3) may also comprise monofunctional hydroxyl-containing compounds. Examples of such monofunctional hydroxyl-containing compounds are ethanol, n-butanol, ethylene glycol monobutyl ether, diethylene glycol monomethyl ether, ethylene glycol monobutyl ether, diethylene glycol monobutyl ether, propylene glycol monomethyl ether, dipropylene glycol monomethyl ether, tripropylene glycol monomethyl ether, dipropylene glycol monopropyl ether, propylene glycol monobutyl ether, dipropylene glycol monobutyl ether, tripropylene glycol monobutyl ether, 2-ethylhexanol, 1-octanol, 1-dodecanol, 1-hexadecanol, etc.

Hydroxyfunctional ionic or potentially ionic hydrophilizing agents as component a4) are taken to mean all compounds which have at least one isocyanate-reactive hydroxyl group and also at least one functionality such as, for example, -COOY, —SO₃Y, -PO(OY)₂ (in which Y⁺ represents, for example, H⁺, NH₄⁺, metal cation), -NR₂, -NR₃⁺ (in which each R individually represents, for example, a hydrogen atom, an alkyl radical, an aryl radical), which, on interaction with aqueous media, enter into a pH-dependent dissociation equilibrium and in this manner can be negatively, positively or neutrally charged.

Suitable ionically or potentially ionically hydrophilizing compounds which correspond to the definition of component a4) include compounds such as, for example, mono- and dihydroxycarboxylic acids, mono- and dihydroxysulfonic acids, and also mono- and dihydroxyphosphonic acids and salts thereof, such as dimethylol propionic acid, dimethylol butyric acid, hydroxypivalic acid, malic acid, citric acid, glycolic acid, lactic acid, the propoxylated adduct of 2-butenediol and NaHSO₃, as is described in, for example, U.S. Pat. No. 4,108,814, the disclosure of which is hereby incorporated by reference, and which is believed to correspond to DE 2,446,440 (see pages 5-9, formulae I-III therein). Also suitable are those compounds which contain, as hydrophilic structural components, for example amine-based building blocks such as, N-methyldiethanolamine, which are convertible into cationic groups.

Preferred ionic or potentially ionic hydrophilizing agents to be used as component a4) herein are those of the abovementioned type which act in a hydrophilizing manner anionically, and preferably via carboxyl or carboxylate and/or sulfonate groups.

Particularly preferred ionic or potentially ionic hydrophilizing agents are those which contain carboxyl and/or sulfonate groups present as anionic or potentially anionic groups. Such compounds include, for example, the salts of dimethylol propionic acid or dimethylol butyric acid.

Suitable nonionically hydrophilizing compounds which can be used as component a4) include, for example, polyoxyalkylene ethers which contain at least one hydroxyl or amino group as an isocyanate-reactive group.

Examples are the monohydroxyfunctional polyalkylene oxide polyether alcohols having a statistical mean of 5 to 70, preferably 7 to 55, ethylene oxide units per molecule. Such monohydroxyfunctional polyalkylene oxide polyether alcohols are accessible in a manner known per se by alkoxylating suitable starter molecules as described, in, for example, Ullmanns Encyclopädie der technischen Chemie [Ullmann's Encyclopaedia of Industrial Chemistry], 4th edition, volume 19, Verlag Chemie, Weinheim, pages 31-38.

These monohydroxyfunctional polyalkylene oxide polyether alcohols are either pure polyethylene oxide ethers or mixed polyalkylene oxide ethers. The mixed polyalkylene oxide ethers preferably contain at least 30 mol %, and more preferably at least 40 mol % of ethylene oxide units, based on 100 mol % of all alkylene oxide units present.

Particularly preferred nonionic compounds are monofunctional mixed polyalkylene oxide polyethers which have from 40 to 100 mol % ethylene oxide units and from 0 to 60 mol % propylene oxide units, with the sum of ethylene oxide units and propylene oxide units totalling 100 mol % of alkylene oxide units present.

Suitable starter molecules for these nonionic hydrophilizing agents are saturated monoalcohols such as, for example, methanol, ethanol, n-propanol, isopropanol, n-butanol, isobutanol, sec-butanol, the isomeric pentanols, hexanols, octanols and nonanols, n-decanol, n-dodecanol, n-tetradecanol, n-hexadecanol, n-octadecanol, cyclohexanol, the isomeric methylcyclohexanols or hydroxymethylcyclohexane, 3-ethyl-3-hydroxymethyloxetane, or tetrahydrofurfuryl alcohol, diethylene glycol monoalkyl ethers such as, for example, diethylene glycol monobutyl ether, unsaturated alcohols such as, for example, allyl alcohol, 1,1-dimethylallyl alcohol or oleyl alcohol, aromatic alcohols such as, for example, phenol, the isomeric cresols or methoxyphenols, araliphatic alcohols such as, for example, benzyl alcohol, anisyl alcohol or cinnamyl alcohol, secondary monoamines such as, for example, dimethylamine, diethylamine, dipropylamine, diisopropylamine, dibutylamine, bis(2-ethylhexyl)amine, N-methyl- and N-ethylcyclohexylamine or dicyclohexylamine, and also heterocyclic secondary amines such as, for example, morpholine, pyrrolidine, piperidine or 1H-pyrazole. Preferred starter molecules are saturated monoalcohols of the abovementioned type. It is particularly preferred to use diethylene glycol monobutyl ether or n-butanol as starter molecules.

Alkylene oxides suitable for the alkoxylation reaction are, in particular, ethylene oxide and propylene oxide, which can be used in the alkoxylation reaction in any desired sequence or else in a mixture.

Suitable compounds to be used as component b1) in accordance with the present invention include, for example, di- or polyamines such as 1,2-ethylenediamine, 1,2- and 1,3-diaminopropane, 1,4-diaminobutane, 1,6-diaminohexane, isophoronediamine, mixtures of isomers of 2,2,4- and 2,4,4-trimethyl-hexamethylenediamine, 2-methylpentamethylenediamine, diethylenetriamine, 1,3- and 1,4-xylylenediamine, α,α,α′,α′-tetramethyl-1,3- and -1,4-xylylenediamine and 4,4-diaminodicyclohexylmethane and/or dimethylethylenediamine. The use of hydrazine or also hydrazides such as adipic dihydrazide is likewise possible.

In addition, component b1) can also comprise compounds which, in addition to a primary amino group, also have secondary amino groups or, in addition to an amino group (primary or secondary), also have OH groups. Examples of these are primary/secondary amines such as diethanolamine, 3-amino-1-methyl-aminopropane, 3-amino-1-ethylaminopropane, 3-amino-1-cyclohexylaminopropane, 3-amino-1-methylaminobutane, alkanolamines such as N-amino-ethylethanolamine, ethanolamine, 3-aminopropanol, neopentanolamine.

In addition, monofunctional amine compounds can also be used as component b1). Some suitable monofunctional amine compounds include, for example, methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)-aminopropylamine, morpholine, piperidine and suitable substituted derivatives thereof, amidoamines of diprimary amines and monocarboxylic acids, monoketimines of diprimary amines, and primary/tertiary amines such as N,N-dimethylaminopropylamine.

Preferably, component b1) is selected from the group consisting of 1,2-ethylenediamine, hydrazine hydrate, 1,4-diaminobutane, isophoronediamine and diethylenetriamine.

Ionically or potentially ionically hydrophilizing compounds which are used as component b2) are taken to include (or mean) all compounds which have at least one isocyanate-reactive amino group and also at least one functionality such as, for example, -COOY, —SO₃Y, -PO(OY)₂ (in which Y represents, for example, H⁺, NH₄⁺, metal cation), -NR₂, -NR₃⁺ (in which each R independently represents, for example, a hydrogen atom, an alkyl group, an aryl group), and which, on interaction with aqueous media, enter into a pH-dependent dissociation equilibrium, and in this manner can be positively, negatively or neutrally charged.

Suitable ionically or potentially ionically hydrophilizing compounds are, for example, mono- and diaminocarboxylic acids, mono- and diaminosulfonic acids and also mono- and diaminophosphonic acids and salts thereof. Examples of such ionic or potentially ionic hydrophilizing agents are N-(2-aminoethyl)-β-alanine, 2- (2-aminoethylamino)ethanesulfonic acid, ethyl enediamine-propylsulfonic or -butylsulfonic acid, 1,2- or 1,3-propylenediamine-β-ethylsulfonic acid, glycine, alanine, taurine, lysine, 3,5-diaminobenzoic acid and the addition product of IPDI and acrylic acid (as described in, for example, EP-A 0 916 647 and particularly as described in Example 1, which is believed to correspond to Canadian Patent Application 2253119,). In addition, use can be made of cyclohexylamino-propanesulfonic acid (CAPS) as described in, for example, WO-A 01/88006, the disclosure of which is hereby incorporated, and which is believed to correspond to U.S. Pat. No. 6,767,958, as a suitable anionic or potentially anionic hydrophilizing agent.

Preferred ionic or potentially ionic hydrophilizing agents for component b2) are those of the abovementioned type which act in a hydrophilizing manner via anionic, and preferably carboxyl groups or carboxylate groups and/or sulfonate groups.

Particularly preferred ionic or potentially ionic hydrophilizing agents for component b2) are those which contain carboxyl and/or sulfonate groups present as anionic or potentially anionic groups. Such compounds include, for example, the salts of N-(2-aminoethyl)-β-alanine, 2-(2-aminoethylamino)ethanesulfonic acid or the addition product of IPDI and acrylic acid (as described in EP-A 0 916 647, Example 1, which is believed to correspond to Canadian Patent Application 2253119).

For the hydrophilization, it is preferred to use a mixture of anionic or potentially anionic hydrophilizing agents and nonionic hydrophilizing agents.

The ratio of NCO groups of the compounds of component a1) to NCO-reactive groups of the components a2) to a4) in the production of the NCO-functional prepolymer is from 1.05:1 to 3.5:1, preferably from 1.2:1 to 3.0:1, and most preferably from 1.3:1 to 2.5:1.

The aminofunctional compounds which are added and reacted with NCO- functional prepolymers are used in an amount such that the equivalent ratio of isocyanate-reactive amino groups of these compounds B) to the free isocyanate groups of the prepolymer is from 40 to 150%, preferably from 50 to 125%, and most preferably from 60 to 120%.

In a preferred embodiment of the invention, anionically and nonionically hydrophilized polyurethane dispersions are used, with their production being made of the components a1) to a4) and b1) to b2) in the following amounts, with the individual amounts totalling 100% by weight:
5 to 40% by weight of component a1),
55 to 90% by weight of component a2),
0.5 to 20% by weight sum of components a3) and b1)
0.1 to 25% by weight sum of components a4) and b2),
based on 100% by weight of the sum of components a1) to a4) and b1) to b2), and
in which from 0.1 to 5% by weight of anionic or potentially anionic
hydrophilizing agents a4) and/or b2) are present.

The % by weight of anionic or potentially anionic hydrophilizing agents a4) and b2) is also based on 100% by weight of the sum of all components a1) to a4) and b1) to b2) which are present in the polyurethane dispersions.

A more preferred embodiment of the invention is one in which the amounts of components a1) to a4) and b1) and b2) are as follows:
5 to 35% by weight of component a1),
60 to 90% by weight of component a2),
0.5 to 15% by weight sum of components a3) and b1),
0.1 to 15% by weight sum of components a4) and b2),
based on 100% by weight of the sum of components al) to a4) and bl) to b2), and in which from 0.2 to 4% by weight of anionic or potentially anionic
hydrophilizing agents a4) and/or b2) are present.

In a most preferred embodiment, the amounts of components a1) to a4) and b1) and b2) are as follows:
10 to 30% by weight of component a1),
65 to 85% by weight of component a2),
0.5 to 14% by weight sum of components a3) and b1),
0.1 to 13.5% by weight sum of components a4) and b2),
based on 100% by weight of the sum of components a1) to a4) and bl) to b2), and in which from 0.5 to 3.0% by weight of anionic or potentially anionic
hydrophilizing agents a4) and/or b2) are present.

In preferred embodiments of the polyurethane dispersions (I), component a1) comprises isophorone diisocyanate and/or 1,6-hexamethylene diisocyanate and/or the isomeric bis(4,4′-isocyanatocyclohexyl)methanes, in combination with a2) polymeric polyols comprising a mixture of polycarbonate polyols and polytetramethylene glycol polyols.

The fraction of polycarbonate polyols in the mixture of polymeric polyols used as component a2) is from 20 to 80% by weight, and from 80 to 20% by weight of polytetramethylene glycol polyols. It is preferred to use a fraction of 30 to 75% by weight of polytetramethylene glycol polyols, and 25 to 70% by weight of polycarbonate polyols. It is more preferred to use a fraction of 35 to 70% by weight of polytetramethylene glycol polyols and 30 to 65% by weight of polycarbonate polyols. In each case, the sum of the percentages by weight of the polycarbonate polyols and of the polytetramethylene glycol polyols totals 100% by weight of the mixture and the fraction of the sum of polycarbonate polyols and polytetramethylene glycol polyether polyols of the total weight of component a2) is at least 50% by weight, preferably 60% by weight, and more preferably at least 70% by weight.

These polyurethane dispersions can be produced in one or more stage(s) in homogeneous or multistage reaction, partially in disperse phase. After polyaddition of a1) to a4), which may be either complete or carried out in part, a dispersion, emulsification or solution step proceeds. Subsequently, if appropriate and/or desired, further polyaddition or modification in disperse phase proceeds.

All methods known from the prior art can be used here such as, for example, prepolymer mixing methods, acetone methods or melt dispersion methods. Preferably, the process proceeds via the acetone method.

For preparation according to the acetone method, for the production of an isocyanate-functional polyurethane prepolymer, customarily components a2) to a4) which must not have any primary or secondary amino groups, and the polyisocyanate component a1), are charged in whole or in part and if appropriate diluted with a solvent which is water-miscible but inert to isocyanate groups, and heated to temperatures in the range from 50 to 120° C. To accelerate the isocyanate addition reaction, the catalysts known in polyurethane chemistry can be added.

Suitable solvents are the customary aliphatic, ketofunctional solvents such as, for example, acetone, 2-butanone, etc. These solvents can be added not only at the start of production, but also, if appropriate, in later parts of the process. Preference is given to acetone and 2-butanone.

Other solvents (i.e. cosolvents) such as xylene, toluene, cyclohexane, butyl acetate, methoxypropyl acetate, N-methylpyrrolidone, N-ethylpyrrolidone, and solvents having ether or ester units, can additionally be used, and completely or partially distilled off. It is also possible for some cosolvents to remain completely in the dispersion such as, in the case of, for example, N-methylpyrrolidone, and N-ethylpyrrolidone.

In one particular embodiment of the invention, the use of cosolvents is avoided completely.

Subsequently, any of components a1) to a4) which are not yet added at the start of the reaction are added.

The reaction of components a1) to a4) to form the prepolymer proceeds partially or completely, but preferably completely. In such a manner, polyurethane prepolymers which contain free isocyanate groups are obtained in the absence of solvent or in solution.

In the neutralization step for the partial or complete conversion of potentially anionic groups to anionic groups, use is made of bases such as tertiary amines including, for example, trialkylamines having from 1 to 12, and preferably from 1 to 6, carbon atoms in each alkyl radical, or alkali metal bases such as the corresponding hydroxides.

Examples of these tertiary amines are trimethylamine, triethylamine, methyldiethylamine, tripropylamine, N-methylmorpholine, methyldiisopropylamine, ethyldiisopropylamine and diisopropylethylamine. The alkyl radicals can also bear, for example, hydroxyl groups, such as in dialkylmonoalkanolamines, alkyldialkanolamines and trialkanolamines. As neutralizing agents, if appropriate, use can also be made of inorganic bases such as aqueous ammonia solution or sodium hydroxide or potassium hydroxide.

Preference is given to ammonia, triethylamine, triethanolamine, dimethylethanolamine or diisopropylethylamine as tertiary amines, and also to sodium hydroxide as a neutralizing agent.

In the case of cationic groups, use is made of dimethyl sulfate or succinic acid or phosphoric acid.

The amount of the bases is from 50 to 125 mol %, preferably between 70 and 100 mol % of the quantity of the substance which contains the acid groups that are to be neutralized. The neutralization can also proceed simultaneously with the dispersion by the dispersion water which already contains the neutralising agent.

Subsequently, in a further method step, if this has not yet proceeded, or only if in part, the resultant prepolymer is dissolved using aliphatic ketones such as, for example, acetone or 2-butanone.

The amine components b1) and b2) can, if appropriate, be used individually or in mixtures in water- or solvent-diluted form in the inventive method, in principle any sequence of addition being possible.

If water or organic solvents are used in conjunction as diluents, the diluent content in the component used for chain extension is preferably 70 to 95% by weight.

Dispersion preferably proceeds subsequent to chain extension. For this, the dissolved and chain-lengthened polyurethane polymer, if appropriate, under severe shear, for example, vigorous stirring, is either charged into the dispersion water, or, vice versa, the dispersion water is stirred into the chain-lengthened polyurethane polymer solutions. Preferably, the water is added to the dissolved chain-lengthened polyurethane polymer.

The solvent still present in the dispersions after the dispersion step is customarily subsequently removed by distillation. It is also possible for removal to proceed even during dispersion.

The residual content of organic solvents in the dispersions essential to the invention is typically less than 1.0% by weight, preferably less than 0.5% by weight, more preferably less than 0.1% by weight, and most preferably less than 0.05% by weight, based on the total weight of the dispersion.

The pH of the dispersions essential to the invention is typically less than 9.0, preferably less than 8.5, and more preferably less than 8.0.

The solids content of the polyurethane dispersion is typically from 20 to 70% by weight, preferably from 30 to 65% by weight, more preferably from 40 to 63% by weight, and most preferably from 50 to 63% by weight. The % by weight of solids is based on 100% by weight of the polyurethane dispersion.

In addition, it is possible to modify the polyurethane-polyurea dispersions (I) which are essential to the invention by polyacrylates. For this embodiment, in the presence of the polyurethane dispersion, an emulsion polymerization of olefinically unsaturated monomers such as, for example, esters of (meth)acrylic acid and alcohols having from 1 to 18 carbon atoms, styrene, vinyl esters and/or butadiene is carried out. as described, for example, in, for example, DE-A-1 953 348 (which is believed to correspond to U.S. Pat. No. 3,705,164), EP-A-0 167 188 (which is believed to correspond to U.S. Pat. No. 4,730,021), EP-A-0 189 945 (which is believed to correspond to U.S. Pat. No. 4,644,030) and EP-A-0 308 115 (which is believed to correspond to U.S. Pat. No. 5,137,961), the disclosures of which are hereby incorporated by reference. The suitable monomers contain one or more olefinic double bonds. In addition, the monomers can contain functional groups such as hydroxyl, epoxide, methylol or acetoacetoxy groups.

In a preferred embodiment of the invention, this modification is omitted.

In principle, it is possible to mix the polyurethane-polyurea dispersions (I) which are essential to the present invention with other aqueous binders. Such aqueous binders can comprise, for example, polyester polymers, polyacrylic polymers, polyepoxy polymers or polyurethane polymers. The combination of radiation-curable binders, as are described, for example, in EP-A-0 753 531, the disclosure of which is hereby incorporated by reference, and which is believed to correspond to U.S. Pat. No. 5,684,081, is also possible. It is likewise possible to blend the polyurethane-polyurea dispersions (I) with other anionic or nonionic dispersions such as, for example, polyvinyl acetate, polyethylene, polystyrene, polybutadiene, polyvinyl chloride, polyacrylates and copolymer dispersions.

In a particularly preferred embodiment of the invention, this modification is omitted.

In the production of the inventive chewable foams, in addition to the polyurethane-urea dispersions (I), these may additionally comprise one or more components selected from the group consisting of (II) one or more foam aids, (III) one or more crosslinkers, (IV) one or more thickeners, (V) one or more aids and (VI) one or more cosmetic additives.

Suitable foam aids to be used as component (II) are commercially conventional aids such as water-soluble fatty acid amides, sulfosuccinimides, hydrocarbon sulfonates, sulfates or fatty acid salts, in which the lipophilic radical preferably contains from 12 to 24 carbon atoms.

Preferred foam aids (II) are alkanesulfonates or sulfates having from 12 to 22 carbon atoms in the hydrocarbon radical, alkylbenzenesulfonates or sulfates having from 14 to 24 carbon atoms in the hydrocarbon radical, or fatty acid amides or fatty acid salts having from 12 to 24 carbon atoms.

The abovementioned fatty acid amides are preferably fatty amides of mono- or di-(C2-C3-alkanol)amines. Fatty acid salts can be, for example, alkali metal salts, amine salts or unsubstituted ammonium salts.

Such fatty acid derivatives are typically based on fatty acids such as lauric acid, myristic acid, palmitic acid, oleic acid, stearic acid, ricinoleic acid, behenic acid or arachidic acid, coconut fatty acid, tallow fatty acid, soya fatty acid and hydrogenation products thereof.

Particularly preferred foam aids (II) are sodium lauryl sulfate, sulfosuccinamides and ammonium stearates, and also mixtures thereof.

Suitable crosslinkers to be used as component (III) include, for example, unblocked polyisocyanate crosslinkers, amide- and amine-formaldehyde resins, phenol resins, aldehyde and ketone resins, such as, for example, phenol-formaldehyde resins, resoles, furan resins, urea resins, carbamic ester resins, triazine resins, melamine resins, benzoguanamine resins, cyanamide resins, or aniline resins.

In a particularly preferred embodiment, the use of crosslinkers is completely omitted.

The thickeners used as component (IV) within the meaning of the present invention are compounds which make it possible to set the viscosity of the resultant mixture of components I through VI in such a manner that production and processing of the inventive foam is promoted. Suitable thickeners include commercially available conventional thickeners such as, for example, natural organic thickeners, for example, dextrins or starch, organic modified natural substances, for example, cellulose ethers or hydroxyethylcellulose, fully organically synthetic thickeners, for example, polyacrylic acids, polyvinylpyrrolidones, poly(meth)acrylic compounds or polyurethanes (associative thickeners), and also inorganic thickeners, for example, bentonites or silicic acids. Preferably, fully organically synthetic thickeners are used as component (IV). It is particularly preferred to use acrylic thickeners which, before addition, may optionally be further diluted with water. Preferred commercially available conventional thickeners are, for example, Mirox® AM (BGB Stockhausen GmbH, Krefeld, Germany), Walocel® MT 6000 PV (Wolff Cellulosics GmbH & Co KG, Walsrode, Germany), Rheolate® 255 (Elementies Specialities, Ghent, Belgium), Collacral® VL (BASF AG, Ludwigshafen, Germany) and Aristoflex® AVL (Clariant, Sulzbach, Germany).

Aids, i.e. component (V), within the meaning of the present invention include, for example, antioxidants and/or light stabilizers and/or other additives such as, for example, emulsifiers, fillers, plasticizers, pigments, silica sols, aluminium, clay, dispersions, flow enhancers or thixotropic agents, etc.

Cosmetic additives, i.e. component (VI), within the meaning of the present invention are, for example, flavorings and aroma substances, abrasives, dyes, sweeteners, etc., and also active ingredients such as fluoride compounds, tooth whiteners, etc.

In accordance with the present invention, the components (II) one or more foam aids, (III) one or more crosslinkers, (IV) one or more thickeners and (V) one or more aids, can each be present in an amount up to 20% by weight, and (VI) one or more cosmetic additives can be present in an amount up to 80% by weight, based on 100% by weight of the foamed and dried chewable foams.

In a preferred embodiment of the present invention, the chewable foams comprise (I) from 80 to 99.5% by weight of the polyurethane dispersion, (II) from 0 to 10% by weight of one or more foam stabilizers, (II) from 0 to 10% by weight of one or more crosslinkers, (IV) from 0 to 10% by weight of one or more thickeners, (V) from 0 to 10% by weight of one or more aids, and (VI) from 0.1 to 20% by weight of one or more cosmetic additives, with the sum being based on the non-volatile fractions of components (I) to (VI), and in which the sum of the individual components (I) to (VI) totals 100% by weight.

In a more preferred embodiment of the present invention, the chewable foams comprise (I) from 80 to 99.5% by weight of the polyurethane dispersion, (II) from 0 to 10% by weight of one or more foam aids, (IV) from 0 to 10% by weight of one or more thickeners, (V) from 0 to 10% by weight of one or more aids, and (IV) from 0.1 to 15% by weight of one or more cosmetic additives, with the sum being based on the non-volatile fractions of components (I) to (VI), and in which the sum of the individual components (I) to (VI) totals 100% by weight.

In a most preferred embodiment of the present invention, the chewable foams comprise (I) from 80 to 99.5% by weight of the polyurethane dispersion, (II) from 0.1 to 10% by weight of one or more foam aids, (IV) from 0.1 to 10% by weight of one or more thickeners, (V) from 0.1 to 10% by weight of one or more aids, and (VI) from 0.1 to 15% by weight of one or more cosmetic additives, with the sum being based on the non-volatile fractions of components (I) to (VI), and in which the sum of the individual components (I) to (VI) totals 100% by weight.

The foam can be produced by introduction of air or the action of corresponding shearing energy (such as, for example, mechanical stirring) or via commercially conventional blowing agents. In the present invention, preference is given to the introduction of air into the chewable foam mixture.

The foamed composition can be applied in the most varied manner to the most varied surfaces or in molds. However, it is preferred to apply the foamed composition by casting, doctor-knife application, rolling, spreading, injecting or spraying.

For shaping, the mixture to be foamed or mixture already foamed can first be placed on a surface or into a mold before it is further processed.

Whereas the foamed material, before drying, has a preferred foam density of 200 to 800 g/l, and more preferably of 200 to 700 g/l, most preferably of 300 to 600 g/l, the density of the resultant inventive gum base after drying is preferably 50 to 600 g/l, and more preferably 100 to 500 g/l.

The foamed material is dried at a temperature between 25 and 150° C., preferably between 30° C. and 120° C., and more preferably at 40 to 100° C. The drying can proceed in a conventional dryer. Drying in a microwave (HF) dryer is also possible.

The inventive chewable foams, after the drying step, will typically have a thickness of 1 mm to 100 mm, preferably 1 mm to 50 mm, and more preferably 1 mm to 30 mm.

The inventive chewable foams can, including in a plurality of layers, for example to produce particularly high foam layers, be applied to the most varied substrates, or cast into molds.

In addition, the inventive foamed compositions can also be used in combination with other support materials such as, for example, textile supports, paper, etc., for example via previous application (for example coating).

The inventive chewable foams possess excellent mechanical properties, and in particular exhibit a high extensibility with high tensile strength. Thus, after the chewing process these chewable foams return to their original shape, have the capacity to clean the chewing surfaces and sides of the teeth, and do not stick to floor coverings.

The following examples further illustrate details for the process of this invention. The invention, which is set forth in the foregoing disclosure, is not to be limited either in spirit or scope by these examples. Those skilled in the art will readily understand that known variations of the conditions of the following procedures can be used. Unless otherwise noted, all temperatures are degrees Celsius and all percentages are percentages by weight.

### EXAMPLES

The solid contents were determined as specified in DIN-EN ISO 3251.

NCO contents were determined, unless explicitly stated otherwise, volumetrically as specified in DIN-EN ISO 11909.

The following substances were used in the examples:
Diaminosulfonate: NH₂-CH₂CH₂-NH-CH₂CH₂-SO₃Na (45% strength in water)
Desmophen® C2200: Polycarbonate polyol having an OH number 56 mg of KOH/g, and a number-average molecular weight 2000 g/mol (commercially available from Bayer MaterialScience AG, Leverkusen, DE)
PolyTHF® 2000: Polytetramethylene glycol polyol having an OH number 56 mg of KOH/g, and a number-average molecular weight 2000 g/mol (commercially available from BASF AG, Ludwigshafen, DE)
PolyTHF® 1000: Polytetramethylene glycol polyol having an OH number 112 mg of KOH/g, and a number-average molecular weight 1000 g/mol (commercially available from BASF AG, Ludwigshafen, DE)
Polyether LB 25: Monofunctional polyether based on ethylene oxide/propylene oxide, having a number-average molecular weight 2250 g/mol, and an OH number 25 mg of KOH/g (commercially available from Bayer MaterialScience AG, Leverkusen, DE)
Stokal® STA: Foam aid based on ammonium stearate, active ingredient content: 30% (commercially available from Bozzetto GmbH, Krefeld, DE)
Stokal® SR: Foam aid based on succinamate, active ingredient content: approximately 34% (commercially available from Bozzetto GmbH, Krefeld, DE)
Mirox AM: Aqueous acrylic acid copolymer dispersion (commercially available from BGB Stockhausen GmbH, Krefeld, DE)
Borchigel ALA: Aqueous, anionic acrylic polymer solution (commercially available from Borchers GmbH, Langenfeld, DE)
Octosol SLS: Aqueous sodium lauryl sulfate solution (commercially available from Tiarco Chemical Europe GmbH, Nuremberg, DE)
Octosol 845: Sodium lauryl sulfate ether (commercially available from Tiarco Chemical Europe GmbH, Nuremberg, DE)

### Example 1

### PUR Dispersion (component I)

144.5 g of Desmophen® C2200, 188.3 g of PolyTHF® 2000, 71.3 g of PolyTHF® 1000 and 13.5 g of Polyether LB 25 were heated to 70° C. Subsequently, at 70° C., over the course of 5 min, a mixture of 45.2 g of hexamethylene diisocyanate and 59.8 g of isophorone diisocyanate was added and the mixture was stirred under reflux until the theoretical NCO value was achieved. The finished prepolymer was dissolved with 1040 g of acetone at 50° C., and subsequently, a solution of 1.8 g of hydrazine hydrate, 9.18 g of diaminosulfonate and 41.9 g of water was added over the course of 10 min. The post-stirring time was 10 min. After addition of a solution of 21.3 g of isophoronediamine and 106.8 g of water, the mixture was dispersed over the course of 10 min. by addition of 254 g of water. Removal of the solvent by distillation in vacuo followed, and a storage-stable dispersion having a solids content of 60.0% was obtained.

### Example 2

### PUR Dispersion (component I)

2159.6 g of a difunctional polyester polyol based on adipic acid, neopentyl glycol and hexanediol (mean molecular weight 1700 g/mol, OH number=66), 72.9 g of Polyether LB 25 (i.e. a monofunctional polyether based on ethylene oxide/propylene oxide (70/30), having mean molecular weight 2250 g/mol, and an OH number 25 mg of KOH/g) were heated to 65° C. Subsequently, at 65° C., over the course of 5 min, a mixture of 241.8 g of hexamethylene diisocyanate and 320.1 g of isophorone diisocyanate was added and stirred at 100° C. until the theoretical NCO value of 4.79% was achieved. The finished prepolymer was dissolved with 4990 g of acetone at 50° C., and subsequently, a solution of 187.1 g of isophoronediamine and 322.7 g of acetone was added over the course of 2 min. The post-stirring time was 5 min. Subsequently, over the course of 5 min, a solution of 63.6 g of diaminosulfonate, 6.5 g of hydrazine hydrate and 331.7 g of water was added. The mixture was dispersed by adding 1640.4 g of water. The solvent was then removed by distillation in vacuo and a storage-stable PUR dispersion having a solids content of 58.9% was obtained.

### Example 3

### PUR Dispersion (component I)

2210.0 g of a difunctional polyester polyol based on adipic acid, neopentyl glycol and hexanediol (mean molecular weight 1700 g/mol, OH number =66) were heated to 65° C. Subsequently, at 65° C., over the course of 5 min, a mixture of 195.5 g of hexamethylene diisocyanate and 258.3 g of isophorone diisocyanate was added and stirred at 100° C. until the theoretical NCO value of 3.24% was reached. The finished prepolymer was dissolved with 4800 g of acetone at 50° C., and subsequently, a solution of 29.7 g of ethylenediamine, 95.7 g of diaminosulfonate and 602 g of water was added over the course of 5 min. The post-stirring time was 15 min. Subsequently, over the course of 20 min, the mixture was dispersed by adding 1169 g of water. The solvent was then removed by distillation in vacuo and a storage-stable PUR dispersion having a solids content of 60% was obtained.

### Example 4

### Production of an Inventive Chewable Foam

1000 g of a commercially available polyurethane dispersion (I) (Impranil DLU, Bayer MaterialScience AG, Germany) were mixed with 15 g of Stokal STA (II), 20 g of Stokal SR (II) and 30 g of Borchigel ALA (IV), and subsequently foamed by introducing air using a hand mixing apparatus. The resultant foam density was 400 g/l. Thereafter, the foamed paste was applied using a film-drawing apparatus consisting of two polished rolls which could be set to an exact distance, and in front of the rear roll, a separation paper was inserted. Using a feeler gauge, the distance between paper and front roll was set. This distance corresponded to the film thickness (wet) of the resultant coating which was selected in such a manner that a dry layer thickness >100 μm was achieved. Subsequently, the material was dried in a drying cabinet at 80° C. for 15 minutes. After taking off the separation paper, the inventive chewable foam was obtained. The performance properties are shown in Table 1.

### Example 5

### Production of an Inventive Chewable Foam

1000 g of the dispersion (I) obtained from Example 1 were mixed with 30 g of Octosol SLS (II), 20 g of Stokal SR (II), 20 g of Octosol 845 (II), 5 g of 5% strength ammonia solution and 15 g of Mirox AM (IV), and subsequently foamed by introducing air using a hand mixing apparatus. The resultant foam density was 400 g/l. Thereafter, the foamed paste was applied using a film-drawing apparatus consisting of two polished rolls which could be set to an exact distance, and in front of the rear roll a separation paper was inserted. Using a feeler gauge, the distance between paper and front roll was set. This distance corresponded to the film thickness (wet) of the resultant coating which was selected in such a manner that a dry layer thickness >100 μm was achieved. Subsequently, the material was dried in a drying cabinet at 80° C. for 15 minutes. After taking off the separation paper, the inventive chewable foam was obtained. The performance properties are shown in Table 1.

TABLE 1
Performance properties of the inventive chewable foams

| Chewable | 100% modulus | Extension | Tensile strength |
|---|---|---|---|
| foam from: | [MPa] | [%] | [MPa] |
| Example 4 | 0.6 | 570 | 2.4 |
| Example 5 | 0.8 | 710 | 5.2 |

The modulus at 100% extension was determined on films having a layer thickness >100 μm.

| Chewable | | |
|---|---|---|
| foam from: | σ_{f}/E | R × E/σ_{f}² |
| Example 4 | 1.4 | 1.5 |
| Example 5 | 1.6 | 1.3 |

σ_{f}: Tensile strength
E: Modulus of elasticity
R: Tear propagation resistance

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

1. A process for cleaning teeth comprising chewing a foam comprising, a polyurethane-polyurea.

2. The process according to claim 1, wherein the foam is not plastically deformable.

3. The process according to claim 1, wherein the foam has a 100% modulus of 0.4 to 5.0 MPa, at a tensile strength of 1 to 55 MPa and an extensibility of 300% to 1800%.

4. The process according to claim 1, wherein the foam has a 100% modulus of 0.1 to 8.0 MPa, at a tensile strength of 0.5 to 80 MPa and an extensibility of 100% to 3000%.

5. The process according to claim 1, wherein the foam has a ratio of tensile strength to modulus of elasticity of greater than or equal to 1 and a ratio of the product of the tear propagation resistance (according to DIN ISO 34-1 (2004)) and the modulus of elasticity to the square of the tensile strength of lower than 4 mm.

6. The process according to claim 1, wherein the foam has a ratio of tensile strength to modulus of elasticity of greater than 1.5 and a ratio of the product of the tear propagation resistance (according to DIN ISO 34-1 (2004)) and the modulus of elasticity to the square of the tensile strength of lower than 1.5 mm.

7. The process according to claim 1, wherein the foam further comprises one or more components selected from the group consisting of one or more foam aids (II), one or more crosslinkers (III), one or more thickeners (IV), one or more aids (V), and one or more cosmetic additives (VI).

8. The process according to claim 1, wherein the polyurethane-polyurea comprise a water dispersion of
A) one or more isocyanate-functional prepolymers which comprise the reaction product of:
a1) one or more organic polyisocyanates, with
a2) one or more polymeric polyols having number-average molecular weights of 400 to 8000 g/mol and OH functionalities of 1.5 to 6, a3) optionally, one or more hydroxyfunctional compounds having molecular weights of 62 to 399 g/mol,
and
a4) optionally, one or more hydroxyfunctional, ionic and/or potentially ionic and/or nonionic hydrophilizing agents;
in which the free NCO groups of A) are reacted in whole or in part with
B) one or more compounds selected from the group consisting of
b1) one or more aminofunctional compounds having molecular weights of 32 to 400 g/mol,
b2) one or more aminofunctional, ionic or potentially ionic hydrophilizing agents,
and
b3) mixtures thereof;
with chain extension, in which the prepolymers A) are dispersed before, during, or after the reaction with said one or more compounds B) in water, and optionally, with potentially ionic groups present which are able to be converted into the ionic form by partial or complete reaction.

9. The process according to claim 8, wherein al) said one or more organic polyisocyanates is selected from the group consisting of 1,6-hexamethylene diisocyanate, isophorone diisocyanate, the isomeric bis-(4,4′-isocyanatocyclohexyl)methanes and mixtures thereof;
and, a2) said one or more polymeric polyols comprises at least 70% by weight, based on 100% by weight of a2), of a mixture comprising (i) one or more polycarbonate polyols and (ii) one or more poly-tetramethylene glycol polyols.

10. The process according to claim 7, wherein the foam aids (II) are present and are water-soluble fatty acid amides, sulfosuccinimides, hydrocarbon sulfonates, sulfates or fatty acid salts.

11. The process according to claim 7, wherein the foam aids (II) are present and are sodium lauryl sulfate, sulfosuccinamides, ammonium steaeates, or mixtures thereof.

12. The process according to claim 7, wherein the crosslinkers (IIII) are present and are unblocked polyisocyanate crosslinkers, amide- and amine-formaldehyde resins, phenol resins, aldehyde and ketone resins, resoles, furan resins, urea resins, carbamic ester resins, triazine resins, melamine resins, benzoguanamine resins, cyanamide resins, or aniline resins.

13. The process according to claim 7, wherein the thickeners (IV) are present and are natural organic thickeners, organic modified natural substances, fully organically synthetic thickeners, and inorganic thickeners.

14. The process according to claim 7, wherein aids (V) are present and are antioxidants, light stabilizers, emulsifiers, fillers, plasticizers, pigments, silica sols, aluminum, clay, dispersions, flow enhancers, or thixotropic agents.

15. The process according to claim 7, wherein cosmetic additives (VI) are present and are flavorings and aroma substances, abrasives, dyes, sweeteners, fluoride compounds, or tooth whiteners.

16. The process according to claim 7, wherein the foam comprises 80 to 99.8% by weight of the polyurethane-urea (I), 0 to 10% by weight of one or more foam stabilizers (II), 0 to 10% by weight of one or more crosslinkers (III), 0 to 10% by weight of one or more thickeners (IV), 0 to 10% by weight of one or more aids (V), and 0.1 to 20% by weight of one or more cosmetic additives (VI), with the sum being based on the non-volatile fractions of components (I) to (VI), and in which the sum of the individual components (I) to (VI) totals 100% by weight.

17. The process according to claim 7, wherein the foam comprise 80 to 99.5% by weight of the polyurethane-urea (I), 0 to 10% by weight of one or more foam stabilizers (II), 0 to 10% by weight of one or more crosslinkers (III), 0 to 10% by weight of one or more thickeners (IV), 0 to 10% by weight of one or more aids (V), and 0.1 to 15% by weight of one or more cosmetic additives (VI), with the sum being based on the non-volatile fractions of components (I) to (VI), and in which the sum of the individual components (I) to (VI) totals 100% by weight.

18. The process according to claim 7, wherein the foam comprise 80 to 99.5% by weight of the polyurethane-urea (I), 0 to 10% by weight of one or more foam stabilizers (II), 0.1 to 10% by weight of one or more crosslinkers (III), 0.1 to 10% by weight of one or more thickeners (IV), 0.1 to 10% by weight of one or more aids (V), and 0.1 to 15% by weight of one or more cosmetic additives (VI), with the sum being based on the non-volatile fractions of components (I) to (VI), and in which the sum of the individual components (I) to (VI) totals 100% by weight.

19. The process according to claim 1, wherein the foam has a thickness of 1 mm to 100 mm.

20. The process according to claim 1, wherein the foam has a thickness of 1 mm to 50 mm.
